(19) United States
(12) Patent Application Publication
(10) Pub. No.: US 2011/0098741 A1
(43) Pub. Date: Apr. 28, 2011

(54) APPARATUS FOR DIAGNOSIS, PERFORMANCE AND/OR REGULATION OF PHYSIOLOGICAL FUNCTIONS, IN PARTICULAR IN AN ANAESTHETIZED PATIENT

(75) Inventor: Ulrich Pfeiffer, Munchen (DE)

(73) Assignee: UP-MED GmbH

(21) Appl. No.: 12/988,292

(22) PCT Filed: Apr. 17, 2009
(86) PCT No.: PCT/EP2009/002837

§ 371 Date: Jan. 10, 2011

(30) Foreign Application Priority Data
Apr. 18, 2008 (DE) 10 2008 019 647.9

(51) Int. Cl. A61B 17/00

(52) U.S. Cl. 606/201

(57) ABSTRACT

The invention relates to an appliance for diagnosis, performance and/or regulation of physiological functions, in particular in an anaesthetized patient, comprising a pressure device (10) for at least one body region or extremity (55) of a patient (50), wherein pressure-increasing means (21) and pressure-reducing means (22) are assigned to the pressure device (10), and a control device (30) is provided with which the pressure in the pressure device (10) can be controlled via the pressure-increasing means (21) and the pressure-reducing means (22).

The invention relates to an apparatus for diagnosis, implementation and/or regulation of physiological functions, in particular an apparatus for regulation of the body temperature, of the peripheral blood volume, of the peripheral blood supply, for examination of heart function and/or for mechanical ventilation, particularly in an anaesthetized patient.

Alone in Germany, eight million operative interventions are currently carried out per year, the vast majority of which on anaesthetized patients. An anaesthesia in almost every form, in particular a general anaesthesia, is however fundamentally not only associated with an elimination of consciousness but also with an accompanying partial elimination of neuro-humoral regulation mechanisms that serve to maintain a normal heart-circulation function and a normal metabolism. Through the general suppression of the central nervous system, this also leads to a reduced sympathicotonia with the following effects:

The vessel tone becomes generally diminished, in particular, however, also in the low-pressure system. As a result of vascular dilatation, this leads to a redistribution of the circulating blood volume from intra-thoracic to extra-thoracic; the venous return-flow and the filling of the heart decrease.

Because of the decreased filling of the heart, the pumped volume falls, as a result of which a blood pressure drop-off is to be observed.

Additionally, a ventilation with positive pressures is, as a rule, carried out with a general anaesthesia, whereby the redistribution of the circulating blood volume from intra-thoracic to extra-thoracic is reinforced.

A compensating increase of the heart frequency is, as a rule, absent because the counter-regulation via the sympathicus is inhibited by the anaesthetic.

Furthermore, the redistribution of the circulating blood volume upon initiation of an anaesthesia causes a reduction of the temperature gradients that normally exist between body exterior and body core. As a consequence thereof, this leads to a central temperature decline, while the temperature in the outer body layers increases slightly. Here, also, the counter-regulation is absent because, as is known, the regulation threshold is displaced to lower temperatures by the anaesthetic. The problem manifests itself often even more pronounced, however, in the re-awoken patient for whom a massive counter-regulation with cold shivers (shivering) etc. suddenly begins because of the reduced core body temperature. Not infrequently, this presents a substantial burden for patients in the awakening phase.

Currently, the standard approach to counteract these effects of a narcosis reside in the infusion of volume, usually in the form of crystalloids (Ringer lactate solution, saline solution, etc.) or also colloidal infusion solutions (e.g. hydroxy-ethyl starch, plasma protein solution, etc.). This approach is based, among other things, on the notion that, through the imperative of pre-operative sobriety, a volume deficit would exist intra-vascularly that would be unmasked through the introduction of the anaesthetic. This assumption has, however, proved to be false. To date, one has routinely refrained from the alternative administration of vaso-constricting (vessel narrowing) substances, as this can also be accompanied by a deterioration of the blood supply in particular organs or vessel areas.

It was therefore the object of the present invention to provide an apparatus for diagnosis, carrying out and/or regulation of physiological functions that avoids the disadvantages of the prior art.

This object is achieved through the apparatus for diagnosis, implementation and/or regulation of physiological functions according to the independent claim. Advantageous embodiments are defined in the dependent claims.

The object is solved in particular by an apparatus for diagnosis, implementation and/or regulation of physiological functions, in particular in an anaesthetized patient, comprising a pressure device (10) for at least one body region or body part, in particular an extremity (55) of a patient 50, wherein pressure increasing means (21) and pressure reducing means (22) are allocated to the pressure device (10) and a control device (30) is provided, with which the pressure in the pressure device (10) is continuously controllable via the pressure increasing means (21) and the pressure reducing means (22). The effects of administering an anaesthetic are thereby at least partially compensated via the apparatus according to the invention. With the apparatus according to the invention, the redistribution of the circulating blood volume can be counteracted by a suitable external compression. Preferably, the patient core temperature is simultaneously thus also held to normal values, or alternatively also targetedly lowered or raised, for example, in that continuously exchanged, temperature-conditioned air or temperature-conditioned fluid is employed as pressure medium. On the other hand, the diagnostic application also arises with the apparatus according to the invention in order to ascertain whether the heart of the patient would react to a volume administration, i.e. whether it is volume-responsive, through which is ascertainable on which part of the Frank-Starling-curve the heart is working and how. Through either the therapeutic or also the diagnostic compression e.g. of the extremities or also of the lower body half, this leads to an increase of the venous return-flow and elevation of the pre-load of the heart, whereby the pumped volume rises with the volume-responsive heart. On the other hand, with the non-volume-responsive heart, the indication for the administration of heart- or circulation-active medications can be provided with the aid of the apparatus.

An apparatus for diagnosis, implementation and/or regulation of physiological functions may particularly be an apparatus for regulation of the body temperature, of the peripheral blood volume, of the peripheral perfusion, for examination of the heart function and/or for mechanical ventilation.

A pressure device (10) is a device with which a pressure, in particular an external pressure, can be applied to at least a part of the extremity of the patient. Preferably, for example, the following two possibilities for the application of external compression to extremities or parts of the body are imaginable: The pressure device (10) may be based upon either the principle of the pressure chamber or a compression. The external compression can thereby be achieved either through a pressure chamber over the extremity or also through materials which envelop the corresponding body parts. Preferably, the pressure device therefore comprises at least one pressure chamber (12) and/or a pressure cuff (11). A pressure chamber is thereby preferably an air-tight chamber for controlled increase and reduction of the air pressure. This may, for example, be achieved with a rigid outer body having air-tight sealable collars. A pressure cuff is a cuff which can be attached to an extremity and then effect an external compression through raising or lowering of an applied pressure. Such a pressure cuff can be configured similar to an article of clothing and may, for example, be configured as pressure-pants or -sleeve or -top. Preferably, the pressure-pants may also encompass the feet, the pressure-legging may also encompass the respective foot, and the pressure-sleeve also the respective hand. Preferably, the pressure device extends over at least 20 cm, particularly preferably over at least 30 cm, and very particularly preferred over at least 40 cm of the body part in the direction of the heart. Preferably, pressures are applied in the pressure device that lie considerably below the arterial pressure. The pressure device is therefore preferably configured so as to apply a pressure of less than the arterial pressure in the corresponding body part. Preferably, the pressure device is therefore configured so as to apply a pressure of less than 20 mmHg, particularly preferably of less than 10 mmHg, above the corresponding venous pressure in the corresponding body part. For this reason, the arterial perfusion of this body part is not blocked.

A body region or a body part is an area or a part of the body of the patient; for example, the upper body. An extremity or a limb is preferably at least a part of the upper extremity (pectoral girdle, arm, hand) or the lower extremity (hip, buttocks, thigh, lower leg, foot). In the foreground at this juncture are the extremities, provided no operations are being carried out on them. It may also particularly relate to the free extremities; for example, arms or legs. The pressure device (10) is preferably configured for at least two extremities. Extremity in the sense of the present invention also constitutes a body half, for example the lower or the upper body half. Preferably, the pressure device (10) is configured for at least one arm and/or at least one leg and/or the chest and/or a body half of the patient. Particularly preferably, the pressure device (10) is configured for at least a part of the body (53) (upper body and/or lower body) of the patient (50). In the extreme case, it is also possible to construct the pressure device for the entire patient and preferably to create access for the region upon which the operation takes place.

The pressure increasing means (21) and the pressure reducing means (22) are devices which can respectively raise or lower the pressure in the pressure device (10). They constitute a type of supply unit for the pressure device. By the changing of the pressure in the pressure device, the externally applied pressure is modified. This can take place through the use of pumps that convey a corresponding medium and thereby alter the externally applied pressure. With the pressure increasing means, the pressure is increased and the compression thereby intensified. The increase may preferably lead to a pressure in the order of the venous blood pressure up to the arterial blood pressure in the corresponding body part. Preferably, the pressure increase amounts to 10 to 20 mmHg. The applied pressure thereby preferably lies between the venous and the arterial blood pressure. In a human, the median venous blood pressure in the body regions concerned lies at about 0 to 30 mmHg and the median arterial blood pressure at about 60 to 140 mmHg.

The previously described procedure in the extremities and, where applicable, in the abdomen, leads primarily to a compression of the extra-thoracic venous system. The ribcage itself cannot be drawn upon for compression of the venous system.

Through the pressure reducing means, the applied pressure is lowered, for example in the corresponding chamber between the chamber inner side and skin of the patient, wherein a strongly raised pressure can be employed in particular structures of the chamber. Preferably, the pressure at the chamber inner side and skin of the patient can be lowered to below the atmospheric pressure. A suction effect is thereby preferably exerted on the chest so that it leads to an enlargement of the chest in the exhalation phase and can thereby effect an inhalation by the patient. In the inhalation phase, the exhalation can—preferably—be passively effected through the retraction force of the chest and lungs through reduction or stopping of the suction effect, or alternatively even a forced (supported) exhalation can be effected through generation of a positive pressure in the chamber inner side.

The control device (so) is preferably a processor or computing unit. The pressure increasing and pressure reducing means can be controlled via the control device. This control device can be configured marketed exclusively for this task; for example, a processor which is exclusively provided and assembled for the apparatus according to the invention, or is adopted through a device which already fulfils other tasks, for example an already available computer, in particular a monitor, which is already being used with the patient. Preferably, the control device is configured to control a pump which respectively introduces or releases a pressure medium via the pressure increasing and pressure reducing means into the pressure device or out of the pressure device, respectively. A storage unit may preferably also be allocated to the control device.

Preferably, the pressure in the pressure device is continuously controllable via the control device. It is thus possible to control the pressure precisely and over the entire time of the operation, as well as the post-operative care or pre-operatively before introduction of a narcosis, respectively. By this continual adaptation of the pressure, a displacement of the blood volume within the patient can be effected. In this way, it is not only possible via the control device to pump up the pressure device or to release medium, but also selectively control the pressure over time.

In a further preferred embodiment of the present invention, an apparatus is provided in which the pressure device (10) comprises more than one pressure segment (15). It is thereby possible to apply the external pressure even more precisely and more variably.

Preferably, the individual pressure segment is independently controllable. In this way, in each pressure segment a separate pressure can be adjusted via the control device which is applied to the region of the extremity of that pressure segment. Particularly preferably, the pressure segments can also be controlled in groups. It is thereby possible to apply a specialized pressure pattern, preferably also to vary same over time or, as the case may be, to apply the same pressure in different pressure segments collected together in a group. It is also conceivable to design the pressure segments collectively controllable, either with the same pressure or with different pressures in the pressure segments at the same time. Preferably, the individual pressure segments are also temperature-conditionable, also differently temperature-conditionable, particularly also variably temperature-conditionable over time. Temperature-conditionable here means the adjustment of a temperature in the pressure segment, preferably by the conditioning of the pressure medium, particularly preferably of air or of water, in the pressure segment of the pressure chamber or the pressure cuff. This may concern the raising or the lowering of the temperature.

The segments can, e.g. with a patient lying on his back, in addition to the horizontal segmentation also be arranged vertically segmented above one another in order to be able to more strongly counteract the gravity-dependent increased accumulation of blood in depending (med: dorsal or posterior) peripheral body regions due to increased pressure in the vertically lower lying pressure segments.

In a further embodiment of the present invention, an apparatus is provided in which the pressure increasing means (21) comprises a fluid or a gas that is introducible into the pressure device (10) and, as the case may be, in which the pressure reducing means (22) comprises a fluid or a gas which is able to drawn out of the pressure device (10). In this way, the pressure in the pressure device can be regulated simply.

As fluid, water comes primarily into consideration. As gas, ambient air is preferably employed. Particularly preferably, an under-pressure or sub-atmospheric pressure (suction) can be adjusted by means of the pressure device thereby also between chamber inner side and skin of the patient, or as the case may be, by the modification of the segmentally applied pressure according to a pressure pattern influence can be exerted on the venous blood distribution in the patient and the venous blood flow in the peripheral body regions concerned.

In a further preferred embodiment of the present invention, an apparatus is provided in which the pressure device (10) is pneumatically or hydraulically controllable. By means of a pneumatic control, for example of the compressive pressure of the segments, the pressure can be particularly simply adjusted.

In a further embodiment of the present invention, an apparatus is provided in which the pressure device (10) is climatically conditionable, in particular physiologically climatically conditionable. Climatically conditionable here means that the temperature and/or the humidity is able to be regulated. This may occur through a climatizing device. This preferably comprises temperature conditioning means or a temperature conditioning device and/or a humidity conditioning device. In this way, it is additionally possible to regulate the exterior body temperature of the patient in the region of the pressure device. Preferably, it is provided that the individual segments of a pressure device are individually climatically conditionable or, as the case may be, temperature conditionable.

In an embodiment, pressure chambers are provided over the lower body half, the arms, as well as over the rip cage, as the case may be. The air (i.e. the medium) in the segments or chambers is pneumatically controllable and physiologically climatically conditioned. All of the chambers are constructed either as re-useable systems, or preferably as single-use systems. The pressure and temperature conditions in each pneumatic chamber are regulated separately or collectively or in groups by a supply and control unit. Before initiation of a narcosis, the body exterior can be warmed by corresponding temperatures in the chambers (by continuous circulation of temperature-conditioned air or temperature-conditioned water) so that a temperature decline in the body core does not arise with the initiation of the narcosis. The problems of intra- and post-operative hypothermia are thereby avoided. On the other hand, the outer body temperature of the patient can preferably be quickly selectively lowered or raised with use of a fluid as pressure means.

In a further embodiment, with initiation of the narcosis, an external compression of extra-thoracic body regions, in particular of the lower body half as well as the arms, can be exerted. Blood is thereby “squeezed” from the low pressure system (venous vessel system) in the direction of the heart and displaced towards intra-thoracic. The “squeezing” of the venous system in this regard preferably takes place peristaltically, for example in a frequency range of 1-4/minute. An additionally applied higher frequency modulation of the basic “squeeze” pressure leads to a vibration and an even better promotion of the arterial as well as also of the venous blood supply. The artificial infusion of liquids for the maintenance of the intra-thoracic blood volume thereby becomes partially or even completely superfluous.

In a further embodiment, a hermetically sealing pressure chamber over the ribcage (thorax) is provided as pressure device. In the non-pressurized condition, the thorax chamber thereby closes sleep-flexibly, hermetically air-tight around the thorax. Pressure conveyance structures (for example pressure tubes) are preferably incorporated into the primary sleep-flexible thorax chamber which, upon filling with pressure medium of a corresponding pressure, assume a stiff, outwardly curving arc-form and thereby generate an under-pressure on the ribcage, which owing to an influx of air or ventilation gas into the lungs leads to an enlargement of the rib cage and thus to an inhalation. By reducing or stopping the suction effect in the inhalation phase, the exhalation can—preferably—be effected passively through the retraction force of the ribcage and the lung, or alternatively even a forced (supported) exhalation is effected through generation of a positive pressure in the chamber inner side.

With respect to the circulation, an intra-thoracic suction effect is also brought about through the enlargement of the thorax as a result of the external suction, which supports the re-distribution of the circulating blood volume from extra- to intra-thoracic. With suitable cyclic control of the pressure in the stiffening system of the thorax chamber, a support or complete ventilation is possible. In this way, the same volumes in the respiratory cycle can be realized with lower positive pressure exertion of the ventilation device and thereby also with low intra-thoracic pressures. In the ideal case, a complete external mechanical ventilation is provided for by external suction on the rib cage according to the principle of the iron lung. In this case, merely an airway safeguard (assurance of free airway access) and an aspiration protection (sealing of the airway for preventing the inhalation of stomach fluid) e.g. via a larynx mask would be sufficient in order to carry out the anaesthesia.

In a further embodiment of the present invention, an apparatus is provided in which the control device (30) is set up to receive signals of at least one sensor (35). In this way, measured signals can be transmitted to the control device for further processing. The control device may determine or apply a corresponding paradigm for regulation of the pressure in the pressure device in dependence upon these measured signals or measured values, as the case may be.

In a further embodiment of the present invention, an apparatus is provided in which the skin temperature, the core temperature, the blood pressure, the pulse-oxymetric oxygen saturation and heart frequency, conductivity and/or humidity is detectible via this at least one sensor. Furthermore, at least one sensor may be provided in the thorax chamber(s) for acquisition of the ECG and thoracic electrical impedance for measurement of the heart frequency, the impedance cardiac output, the impedance thoracic fluid volume, and the impedance breath volume.

In a further embodiment of the invention, an apparatus is provided in which the control device (30) is set up in order to be able to the process venous return-flow and/or pre-load of the heart and/or pumped volume of the heart. These values may thus also be taken into account by the control device for adjustment of the pressure.

In a further embodiment of the present invention, an apparatus is provided in which the control device (30) is set up to adjust the pressure and/or the temperature in the pressure device (10) in dependence upon the signal values of the sensors (35) and/or the values derived there-from; for example, the pulse-pressure variation PPV, the global end-diastolic volume, the intra-thoracic blood volume, the cardiac output, the arterial pressure etc.

In a further embodiment of the present invention, an apparatus is provided in which the control device (30) is set up to control the pressure and/or temperature in the pressure device (10) according to a pressure- or temperature-pattern varying over time, as the case may be. By means of such patterns over time, it is possible to selectively influence the displacement of body fluids, in particular blood. A ventilation of the patient by means of a pattern with a variation of sub-and supra-atmospheric pressure is also realisable. These pressure or temperature patterns, as the case may be, are preferably variable or settable in dependence upon the measured values or the derived values. With change of a measured value it is thus possible to targetedly react through change of the applied pressure pattern. The adjustment control hence preferably takes place online, i.e. in a feedback control loop in response to the measured or derived values.

In a further embodiment of the present invention, an apparatus is provided in which the control device (30) is coupled with a monitor (39). Preferably, it is also possible to couple the supply- and control unit with a monitor. The monitor preferably acquires intra-thoracic liquid and blood volumes as total or in individual compartments. The monitor preferably determines the heartbeat volume and cardiac output and/or continually detects the body core temperature.

In the therapeutic feedback mode, the supply- and control-unit is preferably disposed by means of the monitor to increase the compression pressure on the body compartments optionally pre-selectable by the operator up to a pre-selected maximal pressure (preferably peristaltically vibrating/oscillating) upon decreasing intra-thoracic liquid- and blood-volume and consequently decreasing cardiac output, in order to displace more blood into the thorax again and to elevate the cardiac output. On the other hand, with increasing intra-thoracic fluid- and blood-volumes, the compression pressure can also be lowered or not even applied.

Preferably, the pressure in the individual compression chambers or, as the case may be, in the pressure cuff or the segments, is not tenaciously held constant, but rather a directed control of a pressure wave successively passing through the individual pressure segments/chambers takes place, which contributes to pumping blood essentially peristaltically with oscillating superposition from periphery to centre, for example with a period duration of 15 s. A wave-type compression configured in this manner is able to be even more precisely and effectively controlled, the more individually controllable pressure segments or chambers, for example, the pressure cuff or pressure pant for the lower body region consists of. The wave-type compression may—according to requirements and circulation condition—commence from atmospheric pressure or also a higher pressure and return back to this pressure. Usually, the peak-pressure of the pressure wave to be generated, which means the maximal pressure in the pressure pattern, is selected such that the average venous pressure in the body region to be compressed is thus certainly exceeded, but however certainly lies below the median diastolic arterial pressure. The median pressure of the applied pressure pattern may thereby lie under, equal to, or over the venous pressure of the corresponding body region. The body core temperature can be simultaneously regulated through corresponding heating of the peripheral and lower body- or thorax-pressure chambers via corresponding regulation of the temperature of the air flowing through the chambers under pressure or through additional convective heating by means of other heat sources.

In the diagnostic feedback mode, the monitor preferably induces the supply- and/or control-unit to execute a pre-selectable compression pressure pattern in a pre-selectable time period in pre-selectable body compartments or pressure segments: Preferred is an increase and subsequent decrease of the compression pressure; it may however also be a decrease of the compression pressure with a following increase again.

By means of a diagnostic manoeuvre as previously described, either the intra-thoracic blood volume and thereby the heartbeat volume and cardiac output is temporarily increased, or alternatively also decreased; this only happens however if the heart is volume-responsive. The result of such a manoeuvre is displayed to the operator on the monitor through corresponding parameters such as the pulse-pressure variation PPV, the variation of the photo-plethysmographic pulse signals, through increase of the beat volume and/or of the cardiac output and similar “volume challenge” parameters and assists with the determination whether the pump performance of the heart can be increased by volume administration (infusion solutions or blood) or through administration of heart-strengthening or so-called vaso-active medications.

In a further embodiment of the present invention, an apparatus is provided in which the pressure device (10) comprises at least one sealing collar (13). With such a sealing collar it can be assured that the pressure (essentially) does not escape in the transition from the pressure chamber to the patient or, in the case of the thorax chamber, that the under-pressure generated is not disturbed or weakened by influx of foreign air.

Preferably, with the pressure chamber systems, a hydraulic sealing is provided at the respective transitions. To this end, sealing collars are provided which are configured closely fitting. These sealing collars can, for example, be provided around the hips in the pressure chamber compartment for the lower body half, or on the upper arm in the chambers for the extremities.

In a further embodiment of the present invention, an apparatus is provided in which the at least one sealing collar (13) comprises at least one sensor (35). The sensors accommodated in this fashion thereby lie well against the patient through the close-fitting body contact of the sealing collar.

The sealing collars thus preferably comprise different, non-invasive biosensors, e.g. for ECG, temperature, oscillometric—or other types of blood pressure measurement, sphygmomanometry, pulse-oximetry, electrical impedance tomography and/or electrical impedance cardiography. Preferably, disposable biosensors can be provided in the corresponding disposable cuffs or can already be incorporated, as the case may be.

Preferably, the chamber system in the case of the pressure chamber is constructed such that it hinders the actual operation as little as possible. The chamber system for the lower body half could, for example, therefore also serve as a placement surface for operating instruments, etc. Depending on the construction, it is also advantageous for hygienic reasons to cover the inner space as well as the outer surfaces with hygienically impeccable sterile systems.

As described above, besides the pressure chamber, an external compression can also be achieved through materials which envelope the corresponding body parts. In the present case, a pressure cuff which encompasses the corresponding body part has therefore been described as an alternative preferred embodiment. In the pressure cuff, air is preferably employed as medium and besides the pneumatic control of the compression pressure, a temperature conditioning of the cuff preferably takes place through continuous circulation of temperature-conditioned air in order to avoid heat losses from the body and in order to be able to transfer heat to the body.

All cuffs may either be constructed as re-usable systems, preferably however as disposable systems.

For the lower body half, the pressure cuffs are preferably individually designed for each of the legs (where possible incorporating the feet). Alternatively, the pressure cuffs are designed as pants in the sense of a “pressure pants” which terminates at the level of the waste belt. For the upper body half, individual arm sleeves are also preferably provided (where possible incorporating the hands), in the same way as a “pressure jacket”.

A particular advantage of the pressure cuff resides in the simultaneous, almost ideal protection from bedsores to be attained.

Preferably, a complete body operation suit is provided. This can be put on the patient earlier in the ward. The operation region is preferably rendered sterile beforehand, also in the ward, and is located underneath a transparent window in the operation suit. The sterile covers are preferably integrated in the suit and simply folded away at the start of the operation. All connections of the bio-sensors and the pneumatic/climatic control are preferably configured flange-like. It is thereby possible to achieve an enormous procedural advantage in the perioperative activity.

The invention shall now be more fully explained with reference to drawing figures. In this respect, the following is shown in the figures:

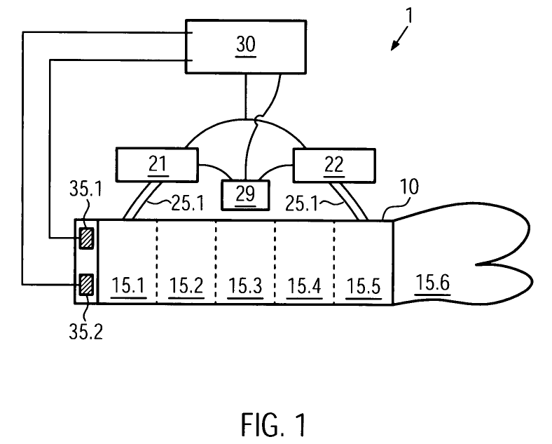

FIG. 1 a schematic view of an embodiment of an apparatus of the present invention for an arm;

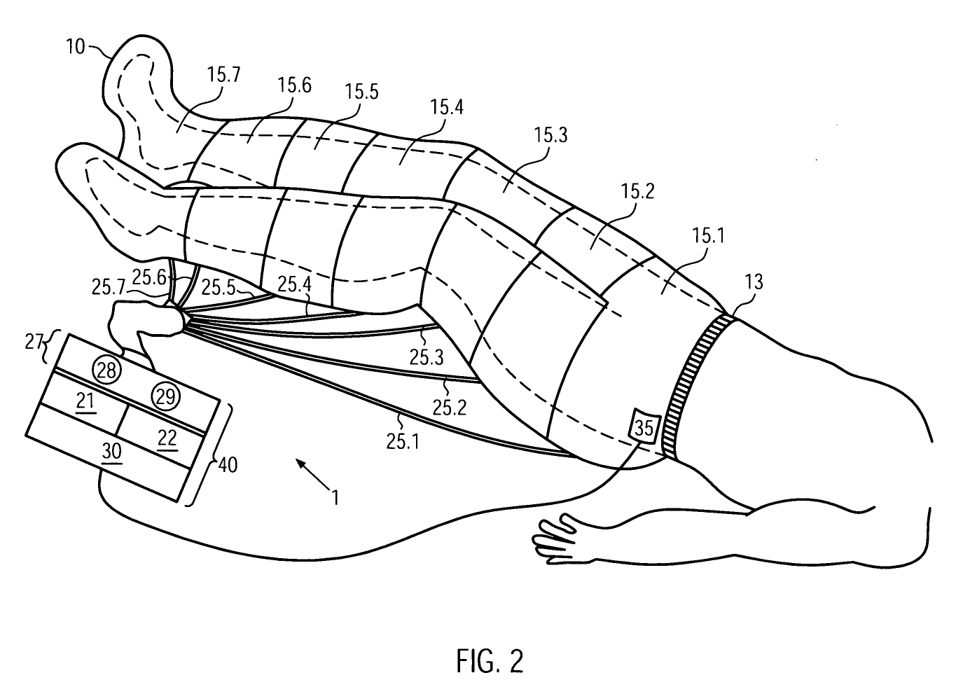

FIG. 2 a view of a further embodiment of the present invention for the legs;

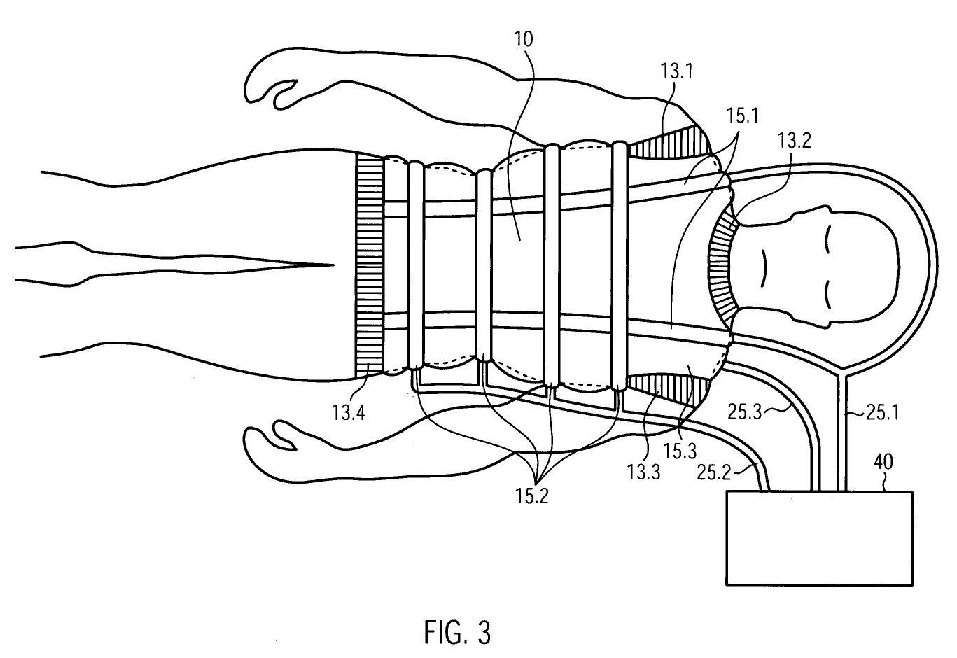

FIG. 3 a view of a further embodiment of the present invention for the ribcage;

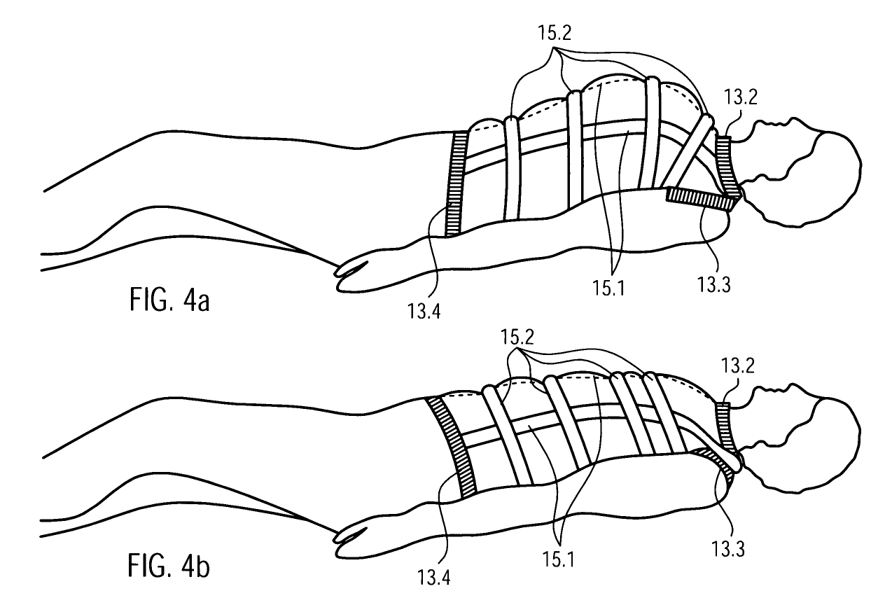

FIG. 4a a further view of the embodiment of FIG. 3 in the inhaled state; and

FIG. 4b a further view of the embodiment of FIG. 3 in the exhaled state.

In FIG. 1 shows a schematic view of an embodiment of an apparatus of the present invention. An apparatus 1 for diagnosis, implementing and/or regulating physiological functions, in particular in an anaesthetized patient, comprises a pressure device 10. This pressure device 10 is configured as a pressure cuff in form of a sleeve for an upper arm with added hand portion in the form a glove. Furthermore, a control device 30 is provided which is connected with a pressure increasing means 21 and a pressure decreasing means 22. The pressure increasing means 21 and the pressure decreasing means 22 are connected with the pressure device 10 via conduits 25.1 and 25.2 respectively. The pressure device 10 consists of six pressure segments 15.1 to 15.6. These pressure segments 15.1 to 15.6 are arranged in regular spacings along the arm to be encompassed by the pressure device such that they represent approximately equal sections adjacent to one another. The first five pressure segments 15.1 to 15.5 are configured as pressure cuffs having cylindrical cross-sections into which the arm can be inserted. The sixth section is configured as pressure segment 15.6 in the form of a glove into which the hand can be inserted. The individual pressure segments 15.1 to 15.6 are individually controllable through the pressure increasing means 21 and the pressure reducing means 22 via the conduits 25.1 and 25.2. Temperature conditioning means 29 are connected to the pressure increasing means 21 and pressure reducing means 22, via which the medium, here air, used in the pressure increasing means 21 and pressure reducing means 22 can be temperature-conditioned. The temperature conditioning means 29 is furthermore connected to the control device 30 so that it can be controlled via the control device 30. Sensors 35.1 and 35.2 are also provided on the pressure cuff. The sensor 35.1 is a temperature sensor and the sensor 35.2 is a sensor for measuring the moisture of the skin.

In operation, an arm of a patient is inserted into the pressure device 10 in the form of a sleeve having the glove section 15.6. The sensors 35.1 and 35.2 are thereby also fixed to the skin. Via the pressure increasing means 21, the pressure segments 15.1 to 15.6 are now adjusted to a pre-determined pressure. This pressure is applied to the arm of the patient. The control device 30 now controls the pressure increasing means 21 and the pressure decreasing means 22 in such a way that a pressure wave is transmitted over the segments 15.6 to 15.1. This happens, for example, in such a way that the pressure in the segment 15.6 is increased and subsequently the pressure in the segment 15.5 is increased and, delayed, in the segment 15.4 is increased while at the same time the pressure in the segment 15.6 is then already reduced again, then the pressure in the segment 15.3 is raised and in 15.5 lowered, then in 15.2 increased and in 15.4 decreased while at the same time the pressure is again increased in 15.6 and from there a second wave is started which follows after the first wave. Blood is thereby displaced from the arm back into the body, as would also be the case with a self-infusion.

The segments 15.1 to 15.6 may also be controlled in groups and the pressure in all of these segments periodically increased and decreased. The blood volume is thereby also displaced in the body.

Optionally, the air or the pressure fluid which is then conveyed into the cuff can be temperature-conditioned via the temperature conditioning device 29. The cuff can thereby be warmed or cooled to regulate the temperature of the arm. Alternatively, it would also be possible that the temperature conditioning device 29 is directly incorporated in the pressure cuff as a heating coil and heat or cooling is electrically or chemically generated there.

FIG. 2 shows a view of a further embodiment of the present invention for the legs. The apparatus for diagnosis, implementing and/or regulating physiological functions 1 comprises a pressure device 10 which is configured in the form of “pants”. These “pants” comprise in each case a legging with sock as well as a section for the hip region. This pressure device 10 is upwardly limited by a sealing collar 13. The “pressure pants” 10 furthermore includes individual pressure segments 15.1 to 15.7 which are arranged adjacent next to one another from the hip region to the sock. The pressure device 10 or the individual pressure segments 15.1 to 15.7, as the case may be, are connected with a regulating device 40 via conduits 25.1 to 25.7. The regulating device 40 comprises pressure increasing means 21 and pressure decreasing means 22, a climate conditioning device 27 as well as a control device 30. The climate conditioning device 27 comprises moisture conditioning means 28 and temperature conditioning means 29. In the region of the sealing collar 13, a sensor 35 is arranged which is fixed to the skin of the patient by the application pressure of the sealing collar 13.

The “pressure pants” 10 are put on the patient and are sealed in the hip region by means of the sealing collar 13 with respect to the skin of the patient such that air is not able to escape from the pressure pants 10. By means of the regulating device 40, a pressure pattern is now applied via the pressure increasing means 21 and the pressure reducing means 22. Ambient air is thereby respectively pressed into or drawn from the individual pressure segments. The pressure segments 15.2 to 15.7 can thereby either be integrated for both legs so that a pressure pattern acts parallel upon the corresponding body regions under the respective pressure segments. It is also conceivable to provide pressure segments 15.2 to 15.7 separately for the right and the left leg and to control the individual pressure segments 15.2 left to 15.7 left as well as 15.2 right to 15.7 right individually and separately from one another. Via the climate conditioning device 27 it is possible to adjust the temperature and the air humidity, respectively, of the air introduced via the conduits 25.1 to 25.7. In this way, the outer temperature of the patient can be regulated. Temperature and moisture of the patient or of the air in the pressure chambers can be ascertained via the sensor 35. This measured value can be fed back to the regulating device in order to be able to re-adjust temperature and moisture via the climate conditioning device 27.

In this way, a displacement of the blood volume is able to be supported. Through peristaltic and rhythmic compression, an infusion administration is thereby able to be avoided. A peripheral perfusion can thereby also be promoted. In addition, an embolism prophylaxis is possible by means of this pressure pants.

FIG. 3 shows a view of a further embodiment of the present invention for the ribcage. A pressure device 10 is provided in the form of a “pressure vest”. At the outlets for the arms, the head, and the termination at the hips, sealing collars 13.1 to 13.4 are provided. The sealing collars are constructed such that they lie on the skin of the patient without exerting their own pressure on the patient. Preferably, these sealing collars are sealed by the use of, for example, gel between the sealing collar and the skin. Pressure segments 15.1 are provided lengthwise over the ribcage in the form of tube-shaped chambers and are securely attached to the pressure vest. Pressure segments 15.2, also in the form of tube-shaped chambers, are provided transversely across the ribcage. These pressure segments 15.2 and 15.2 are thereby preferably constructed for a high pressure, preferably up to one bar, and above all, are longitudinally expandable but not, however, expandable in diameter. They may, for example, be fabricated from silicone so that tubes of this type change length under application of such pressures and thereby lead to an expansion or a reduction of the pressure vest. Also conceivable is that these tube-shaped pressure chambers are attached or stitched gathered onto the vest, i.e. as a kind of concertina-tube. Thus, more material of the tube-shaped pressure segments would then be fixed on the side in contact with the vest per distance than is present on the side facing away from the vest. Upon inflation of concertina structures of this type, the vest would thereby also be expanded according to the type of attachment. When the gathering takes place the other way around, i.e. the tube-shaped element is gathered on the away-facing side, then the vest would be compressed. The pressure vest itself includes at least one pressure segment 15.3 via which pressure can be directly applied to the surface of the patient in the region of the vest. The individual pressure segments 15.1 to 15.3 are connected via tube conduits 25.1 to 25.3 with the regulating device 40—composed, for example, like the one in FIG. 2.

In operation, it is then possible to introduce or, as the case may be, to withdraw pressurized air in the pressure segments 15.1 to 15.3 via the conduits 25.1 to 25.3. Similarly, as with the embodiments of FIGS. 1 and 2, a pressure pattern can again be exerted on the patient and the body temperature can be regulated via the pressure segments 15.3. The pressure lies in this respect in the venous pressure range or slightly above (0-30 Torr) respectively. The pressure segment 15.3 is thereby rhythmically and vibratingly charged with pressure.

The breathing of the patient can be supported or even independently carried out by means of a corresponding pressure charging via the tube-shaped configured pressure segments 15.1 and 15.2, respectively. This shall be more precisely explained with reference to the FIGS. 4a and 4b.

FIG. 4a shows a further view of the embodiment from FIG. 3 in the inhaled state. The conduits and the regulating device from FIG. 3 are not illustrated here once again.

Pressurized air has now been introduced into the longitudinally-only expandable pressure segments 15.2 and these thereupon expand. The pressure segments 15.1 are pressure-less or even supplied with sub-atmospheric pressure in the process, which effects a shortening, and in total, an erection of the structure similar to the change of the rib position resulting from spontaneous inhalation. By virtue of this expansion and the secure connection with the pressure vest, the vest is expanded and thereby exerts a suction effect on the ribcage. The ribcage rises and the patient simultaneously breathes in.

FIG. 4b shows a further view of the embodiment from FIG. 3 in the exhaled state.

Here, the pressurized air from FIG. 4a is now released and the tube-shaped pressure segments 15.2 contract again (for increased speed or “forced breathing” these may also be contracted with sub-atmospheric pressure), the pressure segments 15.1 are simultaneously pressure charged, which in total—as with spontaneous breathing—leads to lowering of the ribcage. This is preferably supported through a corresponding material selection for these tube-shaped pressure segments 15.1 and 15.2; for example, in that they are formed from an elastic, only longitudinally expandable material and are pre-stretched such that they exert a slight pressure-effect on the ribcage in the relaxed state of FIG. 4b.

In this way, it is possible to artificially ventilate a patient with the embodiment according to the FIGS. 3, 4a and 4b.

### REFERENCE NUMERAL LIST

1 Apparatus for diagnosis, carrying out and/or regulating physiological functions
10 Pressure device
11 Pressure cuff
12 Pressure chamber
13 Sealing collar
15 Pressure segment
21 Pressure increasing means
22 Pressure reducing means
23 Conduit
27 Climatic conditioning device
28 Humidity conditioning means
29 Temperature conditioning means
30 Control device
35 Sensor
40 Regulating device
50 Patient
53 Body of the patient
55 Extremities of the patient 50

1. Apparatus for diagnosis, implementing and/or regulating physiological functions, in particular in an anaesthetized patient, comprising a pressure device for at least one body region of a patient, wherein pressure increasing means and pressure reducing means are allocated to the pressure device and a control device is provided, with which the pressure in the pressure device is continuously controllable via the pressure increasing means and the pressure reducing means.

2. Apparatus according to claim 1, wherein the pressure device is configured for at least one arm and/or at least one leg and/or the ribcage and/or the lower body half of the patient.

3. Apparatus according to claim 1, wherein the pressure device is configured for at least two extremities.

4. Apparatus according to claim 1, wherein the pressure device is configured for at least one part of the body of the patient.

5. Apparatus according to claim 1, wherein the pressure device includes more than one pressure segment.

6. Apparatus according to claim 1, wherein the pressure increasing means comprises a fluid or a gas which is able to be supplied to the pressure device.

7. Apparatus according to claim 1, wherein the pressure reducing means comprises a fluid or a gas which is able to be drawn from the pressure device.

8. Apparatus according to claim 1, wherein the pressure device is pneumatically controllable.

9. Apparatus according to claim 1, wherein the pressure device is climatically controllable.

10. Apparatus according to claim 1, wherein the control device is set up to receive signals from at least one sensor.

11. Apparatus according to claim 10, wherein skin temperature, core temperature, blood pressure, conductivity and/or moisture is detectable via the at least one sensor.

12. Apparatus according to claim 1, wherein the control device is set up to be able to process venous return-flow and/or pre-load of the heart and/or beat volume of the heart.

13. Apparatus according to claim 1, wherein the control device is set up to adjust the pressure and/or temperature in the pressure device in dependence upon the signal values of the sensors and/or values derived there-from.

14. Apparatus according to claim 1, wherein the control device is set up to regulate the pressure and/or the temperature in the pressure device according to a pressure or temperature pattern varying over time.

15. Apparatus according to claim 1, wherein the control device is coupled with a monitor.

16. Apparatus according to claim 1, wherein the pressure device includes at least one sealing collar.

17. Apparatus according to claim 16, wherein the at least one sealing collar includes at least one sensor.

18. Apparatus according to claim 1, wherein the pressure device includes at least one pressure sleeve and/or a pressure chamber.
